(19) United States
(12) Patent Application Publication
(10) Pub. No.: US 2004/0052733 A1
(43) Pub. Date: Mar. 18, 2004

(54) Pharmaceutical compositions for inhalation

(76) Inventors: John Nicholas Staniforth, Bath (GB); David Alexander Vodden Morton, Bath (GB)

Correspondence Address: DAVIDSON, DAVIDSON & KAPPEL, LLC, 485 SEVENTH AVENUE, 14TH FLOOR, NEW YORK, NY, 10018, US

(21) Appl. No.: 10/433,138

(22) PCT Filed: Nov. 15, 2001
(86) PCT No.: PCT/GB01/05056

(30) Foreign Application Priority Data
Nov. 30, 2000 (GB) 0029265.6
Nov. 30, 2000 (GB) 0029263.1
Nov. 30, 2000 (GB) 0029261.5
Nov. 14, 2001 (GB) 0127359.8

(51) Int. Cl. A61L 9/04; A61K 9/14; A61K 31/56

(52) U.S. Cl. 424/46; 514/171

(57) ABSTRACT

The invention relates to compositions for inhalation comprising active particles and cholesterol particles. The compositions may be for use in a pressurised metered dose inhaler or for use in a dry powder inhaler. The invention also relates to an excipient composition comprising particles of excipient material and cholesterol.

The present invention relates to compositions for inhalation.

It is known to administer to patients drugs in the form of fine particles (active particles). For example, in pulmonary administration a particulate medicament composition is inhaled by the patient. Pulmonary administration is particularly suitable for medicaments which are intended to cure or alleviate respiratory conditions such as asthma and for medicaments which are not suitable for oral ingestion such as certain biological macromolecules. Known devices for the administration of drugs to the respiratory system include pressurised metered dose inhalers (pMDI's) and dry powder inhalers (DPI's).

The size of the active particles is of great importance in determining the site of the absorption. In order that the particles be carried deep into the lungs, the particles must be very fine, for example having a mass median aerodynamic diameter of less than 10 μm. Particles having aerodynamic diameters greater than 10 μm are likely to impact the walls of the throat and generally do not reach the lung. Particles having aerodynamic diameters in the range of 5 μm to 0.5 μm will generally be deposited in the respiratory bronchioles whereas smaller particles having aerodynamic diameters in the range of 2 to 0.05 μm are likely to be deposited in the alveoli.

Such small particles are, however, thermodynamically unstable due to their high surface area to volume ratio, which provides significant excess surface free energy and encourages particles to agglomerate. In the inhaler, agglomeration of small particles and adherence of particles to the walls of the inhaler are problems that result in the active particles leaving the inhaler as large agglomerates or being unable to leave the inhaler and remaining adhered to the interior of the inhaler.

In an attempt to improve that situation, dry powders for use in dry powder inhalers often include particles of an excipient material mixed with the fine particles of active material. Such particles of excipient material may be coarse. Coarse excipient particles are referred to as carrier particles and may, for example, have mass median aerodynamic diameters greater than 90 μ. Alternatively, the excipient particles may be fine.

The step of dispersing the active particles from each other and from particles of excipient material, if present, to form an aerosol of fine active particles for inhalation is significant in determining the proportion of the dose of active material which reaches the desired site of absorption in the lungs. In order to improve the efficiency of that dispersal it is known to include in the composition additive materials. Such additive materials are thought to reduce the attractive forces between the particles thereby promoting their dispersal. Compositions comprising fine active particles and additive materials such as amino acids and phospholipids are disclosed in WO 97/03649. Compositions comprising fine active particles, additive materials and carrier particles are disclosed in WO 96/23485.

It has also long been desired to develop pharmaceutical formulations in which the pharmaceutically active substance is released over a comparatively long period of time in order to maintain the concentration of the active substance in the body at a desired level for a comparatively longer period of time. An associated benefit is an increase in patient compliance with the dosing regime brought about by reducing the number of, and/or the frequency of, the administrations necessary to maintain the concentration of the active substance in the body at the desired level.

Delayed release compositions have been developed for delivery of drug to the gastrointestinal tract and some such compositions are commercially available. Systems for the controlled delivery of an active substance through the skin have also been developed.

There remains a need to develop a delayed release composition for pulmonary administration having satisfactory properties.

The present invention provides a composition for use in an inhaler comprising active particles and cholesterol.

The cholesterol has been found to promote the dispersal of the active particles to form an aerosol which is then inhaled by the patient. That process takes place upon actuation of the inhaler and, as noted above, is thought to be a key step in determining the proportion of the dose of active material which reaches the desired site of absorption in the lungs and is especially important where the inhaler is a dry powder inhaler. The cholesterol is believed to reduce the forces of attraction between the particles, although that belief should not in any way be taken as limiting the invention.

It also is believed that the cholesterol, by virtue of its hydrophobic nature, can act as a barrier between aqueous fluids and the active particles, especially in compositions where cholesterol is present on the surface of the active particles, thereby reducing the rate of absorption of the active substance into the body. The composition of the invention therefore may be able to release the active substance over a longer period than a composition comprising similarly-sized particles of the active substance in the absence of cholesterol and therefore a reduced frequency of administration, preferably only once a day or less, may be possible. Furthermore, any delayed release of the active substance may provide a lower initial peak of concentration of the active substance which may result in reduced side effects associated with the active substance.

A delayed release effect due to the presence of cholesterol in the compositions will be of particular value where the active substance is one which exerts its pharmacological effect over a limited period and where, for therapeutic reasons, it is desired to extend that period. The duration of the pharmacological effect for any particular active substance can be measured by methods known to the skilled person and will be based on the administration of the dose of that substance that is recognised as being optimal for that active substance in the circumstances. For example, where the active substance is salbutamol sulphate, the duration of the pharmacological effect will be measured by measuring the effect of administering a dose of the medically-recommended quantity of salbutamol upon the patients' respiratory volume. The means of measuring the duration of the period over which a particular active substance exerts its pharmacological effect will depend upon the nature of the active substance and may include, for example, the monitoring of variables relating to inhalation such as FEV₁ level where the active substance is one which exerts a pharmacological effect over the pulmonary system, for example, salbutamol. Further examples include the monitoring of blood sugar levels where the active substance is insulin or the subjective monitoring of pain relief by the patient where the active substance is an analgesic. Where it is not possible to unambiguously monitor the duration of the pharmacological effect of the active substance, for example, because that duration depends from instance to instance upon external factors beyond experimental control, the duration of the pharmacological effect may be assumed to be the same as the duration over which the active substance has the desired concentration in a relevant bodily fluid. Methods for measuring such concentrations are known to the skilled person. Advantageously, the composition is such that the active substance exerts its pharmacological effect over a period of at least 12 hours, more advantageously at least 15 hours, preferably at least 24 hours.

The delayed release effect of the cholesterol will be greater in compositions comprising a relatively large amount of cholesterol and in compositions where the cholesterol is associated with the surfaces of the active particles. Preferably, the composition is such that the rate of dissolution of the active substance (when tested according to the procedure given below) is no greater than 80%, more preferably no greater than 70%, advantageously no greater than 50% and most preferably no greater than 30%, of the rate of dissolution of particles of the active substance.

The composition may also comprise a propellant and be suitable for use in a pMDI.

Preferably, the composition is a powder for use in a dry powder inhaler.

The powder may comprise at least 10%, advantageously at least 40%, preferably at least 70% and may comprise at least 90% by weight of the active particles. Alternatively, especially where the powder comprises carrier particles, the powder may comprise less than 50%, preferably less than 30%, more preferably less than 10% and in some cases less than 2% by weight of the active particles.

The composition may comprise not more than 90%, advantageously not more than 50%, preferably not more than 30%, more preferably not more than 20% and may in some cases comprise not more than 10% by weight of cholesterol based on the weight of the total composition. The composition will usually comprise at least 0.01% of the cholesterol by weight based on the total weight of the composition.

Preferably, the cholesterol is present in the form of particles. The cholesterol particles will advantageously have a mass median aerodynamic diameter (MMAD) of 50 μm or less, preferably 10 μm or less, more preferably 5 μm or less, especially preferably 2 μm or less and most preferably 1 μm or less. As cholesterol is a relatively soft material, it is difficult to produce relatively fine cholesterol particles using conventional milling techniques. Ball milling, centrifugal ball milling, jet milling and bead milling may provide cholesterol particles having an MMAD of less than 10 μm, especially when carried out at a low temperature. Particles of cholesterol having an MMAD below 4 μm, for example, of 2 μm or less, may be provided by a homogenization technique in which a suspension of cholesterol particles is forced through an orifice under pressure. Shear forces on the particles, impacts between the particles and machine surfaces or other particles and cavitation due to acceleration of the fluid may all contribute to the fracture of the particles. The production of the particles of cholesterol may therefore involve the use of shear forces in a liquid medium. Suitable homogensiers include the EmulsiFlex high pressure homogeniser which is capable of pressure up to 4000 Bar, Niro Soavi high pressure homogenisers (capable of pressures up to 2000 Bar), and the Microfluidics Microfluidiser (maximum pressure 2750 Bar). The homogenisation may be carried out at a pressure of at least 10,000 psi, preferably at least 20,000 psi. The homogenisation may be carried out for at least 60 minutes.

The liquid medium used in the homogenisation is preferably one in which cholesterol is substantially insoluble. The homogenisation may, however, be carried out in a liquid in which the cholesterol is soluble to a limited extent as long as the the amount of cholesterol that is present is such that not all of the cholesterol is dissolved. Separation of the cholesterol particles from the liquid may involve evaporation of the liquid, optionally followed by a drying stage, optionally followed by a brief milling step to break up any agglomerated mass or cake of cholesterol particles.

The homogenization or milling of the cholesterol particles may be conducted in the absence of other components of the composition. However, it may be advantageous to carry out milling or homogenization of cholesterol in the presence of particles of excipient material and/or particles of active material as described below.

Other methods which may be suitable for making fine cholesterol particles include:

i) freeze drying;

ii) spray drying;

iii) spray freeze drying;

iv) gas phase condensation, that is, condensation of a vapour of cholesterol into cholesterol particles; and

v) supercritical fluid methods.

In a further aspect, the invention provides particles of cholesterol having a diameter of 2 μm or less. Preferably, the particles of cholesterol have a diameter of 1.5 μm or less, advantageously 1 μm or less and most advantageously 0.8 μm or less.

A variety of differently defined diameters are known to those skilled in the art of inhaler aerosols. As used herein, unless the context demands otherwise, the word “diameter” may refer to any one of the following known definitions of diameter.

i) Mass Median aerodynamic diameter (MMAD). The MMAD of particles of cholesterol of the present invention is determined using Multi-Stage Liquid Impinger in accordance with the method described in European Pharmacopoeia (supplement 2000) 2.9.18. (Aerodynamic assessment of fine particles) for powder inhalers.

ii) Volume equivalent diameter (VED). The VED is the diameter of a sphere having the same volume as that of the particle of additive material. The VED and methods of measuring the VED are discussed in Aerosol Technology, Properties, Behaviour and Measurement of Airborne Particles, Second Edition, William C. Hinds, Wiley-Interscience, John Wiley & Sons (see especially pages 51 and 52 and 402 to 408). For the purposes of the invention, the particles of cholesterol are regarded as having a specified VED, for example 2 μm, if 90% (by number) of those particles have a VMD of less than that specified length.

iii) Volume median diameter (VMD). Volume median diameters can be measured by laser light scattering.

iv) Stokes Diameter. The Stokes diameter is the diameter of the sphere which has the same density (density of the bulk material) and the same falling velocity as the particle of cholesterol. The Stokes diameter is discussed in Physical and Chemical Properties of Aerosols, Ed. I. Colbeck, Blackie Academic and Professional, Chapman & Hall. For the purposes of the invention, the particles of cholesterol are regarded as having a Stokes diameter of not more than a specified length, for example 2 μm, if 90% of those particles have a Stokes diameter of less than that specified length.

For the avoidance of doubt, particles of cholesterol are regarded as being within the scope of the invention if they have a diameter of not more than 2 μm according to any one of the definitions i) to iv) given above. Thus, particles of cholesterol are within the scope of the invention if they have a MMAD, a VED, a VMD or a Stokes diameter of less than 2 μm. The preferred measure is however MMAD and accordingly, preferably, the MMAD of the particles of cholesterol is not more than 2 μm, advantageously not more than 1.5 μm, more advantageously not more than 1 μm and most advantageously not more than 0.8 μm.

The composition may also contain particles of an excipient material, for example, the composition may contain fine excipient particles and/or carrier particles. Preferably, especially where the composition is a powder for use in a dry powder inhaler, the composition comprises fine excipient particles. The term “fine excipient particles” as used herein refers to particles of excipient material having a mass median aerodynamic diameter (MMAD) of less than 20 μm. Preferably, the fine excipient particles have a MMAD of not more than 15 μm, advantageously not more than 10 μm and preferably not more than 5 μm. The fine excipient particles will in general have an MMAD of at least 0.1 μm. Furthermore, 90% by weight of the fine excipient particles may have a diameter of less than 50 μm, advantageously less than 20 μm, more preferably less than 15 μm, more advantageously less than 10 μm and especially advantageously less than 5 μm.

Volume median diameters and measurements of the proportion of particles having a diameter less than a certain value may be determined by the Malvern laser light scattering method.

Where the composition is a powder for use in a dry powder inhaler, the composition may comprise carrier particles. Such carrier particles are relatively large particles of excipient material.

Advantageously, substantially all (by weight) of the carrier particles have a diameter which lies between 20 μm and 1000 μm, more preferably 50 μm and 1000 μm. The diameter of substantially all (by weight) of the carrier particles may be less than 355 μm and may lie between 20 μm and 250 μm. At least 90% by weight of the carrier particles may have a diameter between from 60 μm to 180 μm. The MMAD of the carrier particles may be in the range of from 20 μm to 250 μm. Carrier particles of such size are known for use in powders for inhalation. The relatively large diameter of the carrier particles improves the opportunity for other, smaller particles to become attached to the surfaces of the carrier particles and to provide good flow and entrainment characteristics and improved release of the active particles in the airways to increase deposition of the active particles in the lower lung.

The proportions in which the carrier particles (if present), fine excipient particles (if present), cholesterol and active particles and any other components are mixed will, of course, depend on the type of inhaler device used, the type of active particles used and the required dose.

Recently, formulations have been developed having larger carrier particles, referred to herein as “large carrier particles”. The composition may comprise large carrier particles having a mass median diameter (MMD) of at least 175 μm. In fact, it is preferred that the MMD of the large carrier particles is at least 200 μm, and more preferably at least 250 μm.

The large carrier particles may have a volume diameter of at least 50 μm. Although the formulation may include particles of volume diameter less than 50 μm of the same material as the carrier particles, those smaller particles are not included within the term “large carrier particles” as used herein. Advantageously, not more than 10% by weight, and preferably not more than 5% by weight, of the large carrier particles have a volume diameter of 150 μm of less. Advantageously at least 90% by weight of the large carrier particles have a volume diameter of 175 μm or more, and preferably 200 μm or more. Advantageously, at least 90% by weight, and preferably at least 95% by weight, of the large carrier particles have a volume diameter of not more than 1 mm. Preferably at lest 90% by weight of the large carrier particles have a volume diameter of not more than 600 μm. Advantageously, at least 50% by weight, and preferably at least 60% by weight, of the large carrier particles have a volume diameter between 150 μm and 750 μm, more preferably between 150 μm and 650 μm. Particular advantages are offered by formulations in which substantially all of the large carrier particles have a volume diameter in the range of about 210 to about 360 μm or from about 350 to about 600 μm.

The carrier particles may be of any acceptable pharmacologically inert material or combination of materials. For example, the carrier particles may be composed of one or more materials selected from sugar alcohols; polyols, for example sorbitol, mannitol and xylitol, and crystalline sugars, including monosaccharides and saccharides; inorganic salts such as sodium chloride and calcium carbonate; organic salts such as sodium lactate; and other organic compounds such as urea, polysaccharides, for example starch and its derivatives; oligosaccharides, for example cyclodextrins and dextrins. Advantageously the carrier particles are of a crystalline sugar, for example, a monosaccharide such as glucose or arabinose, or a disaccharide such as maltose, saccharose, dextrose or lactose. Preferably, the carrier particles are of lactose.

The large carrier particles preferably have a fissured surface, that is, on which there are clefts and valleys and other recessed regions, referred to herein collectively as fissures. The fissures should preferably be at least 5 μm wide extending to at least 5 μm deep, preferably at least 10 μm wide and 10 μm deep and most preferably at least 20 μm wide and 20 μm deep.

Because of the excellent flow properties of formulations containing those large fissured carrier particles, such formulations offer special advantages in the administration of active agents to be administered in relatively large doses. Moreover, the fissured carrier particles offer particular advantages in that they are capable of retaining relatively large amounts of fine material for example, active particles and particles of cholesterol, in the fissures without or with only little segregation.

A number of methods may be used to determine whether carrier particles have a fissured surface that will offer the above-mentioned capability of retaining relatively large fines contents substantially without segregation. Those methods are described in International Patent Application No. PCT/GB01/01732.

The large carrier particles are advantageously in the form of an agglomerate consisting of a plurality of crystals fused to one another, the fastness of agglomeration being such that the carrier particles have substantially no tendency to disintegrate on expulsion from the inhaler device. In the case of crystalline sugars, such as lactose, such structures may be obtained in a wet granulation process, in which crystals within an agglomerate become fused to one another by solid bridges, the resultant structure having a complex shape of high irregularity and/or high fractal dimension, including a multiplicity of clefts and valleys, which in some cases may be relatively deep.

Suitably shaped carrier particles also include dendritic spherulites of the type disclosed in U.S. Pat. No. 4,349,542 for use in tablet manufacture.

Where carrier particles are included in the pharmaceutical composition, fine excipient particles may also present in an amount of from 1% to 40%, more preferably 5% to 20% based on the weight of the carrier particles. The carrier particles are preferably present in an amount of at least 50%, more preferably 60%, advantageously 75% based on the combined weight of the active particles, the fine excipient particles, the cholesterol and the carrier particles.

Where the pharmaceutical composition is a powder for use in a dry powder inhaler and does not comprise carrier particles the fine excipient particles may be present in an amount of at least 1%, more preferably at least 5%, advantageously at least 10% and most preferably at least 20% by weight based on the combined weights of the fine excipient particles, the cholesterol and the active particles. The fine excipient particles will preferably be present in an amount of not more than 95%, more preferably not more than 90% and especially advantageously not more than 70% based on the combined weights of the fine excipient particles, the cholesterol, and the active particles.

In order for the cholesterol to promote the dispersal of the particles in the composition to form an aerosol upon actuation of an inhaler, it is important that the cholesterol is present in the composition as a discrete phase (e.g. in the form of particles) rather than being present as part of a mixture with the other components of the composition on a molecular level, for example, as a solid solution in those other components. Preferably, the cholesterol is not present in the form of liposomes or as a component in liposomes. At least some and preferably a major proportion of the cholesterol is present in the form of particles attached to or as a coating on the surfaces of other particles present in the composition. Preferably, at least some of the cholesterol is present on the surfaces of the active particles. Where the active particles form a substantial proportion of the composition and the fine excipient particles and/or carrier particles are not present or are present only in minor amounts (less than 50% by weight of the composition), advantageously at least 30%, preferably at least 40% and most preferably at least 60% of the cholesterol is present on the surfaces of the active particles. Where the composition also contains other particles such as fine excipient particles and carrier particles, cholesterol may also be present on the surfaces of those other particles. In particular, at least some of the cholesterol may be present on the surfaces of the fine excipient particles and/or the carrier particles.

The cholesterol may be present as a coating, which may be a partial or discontinuous coating, on the surfaces of active particles and/or particles of excipient. Such a coating may be formed by applying a solution of the cholesterol in a volatile solvent to the particles and then drying to remove the solvent. Preferably, however, the cholesterol is present in the form of particles. 90% (by number) of the particles of cholesterol will preferably have diameters (as determined by microscopy) of less than 50 μm, advantageously less than 20 μm, more advantageously less than 10 μm, more preferably less than 5 μm and especially preferably less than 2 μm and in some cases less than 1 μm. The particles of cholesterol will generally have diameters of at least 0.01 μm.

The terms “active particles” and “particles of active material” are used interchangeably herein. The active particles referred to throughout the specification will comprise one or more pharmacologically active agents. The active particles advantageously consist essentially of one or more pharmacologically active agents. Suitable pharmacologically active agents may be materials for therapeutic and/or prophylactic use. Active agents which may be included in the formulation include those products which are usually administered orally by inhalation for the treatment of disease such as respiratory disease, for example, β-agonists.

The active particles may comprise at least one β₂-agonist, for example one or more compounds selected from terbutaline, salbutamol, salmeterol and formoterol. If desired, the active particles may comprise more than one of those active agents, provided that they are compatible with one another under conditions of storage and use. Preferably, the active particles are particles of salbutamol sulphate. References herein to any active agent is to be understood to include any physiologically acceptable derivative. In the case of the B₂-agonists mentioned above, physiologically acceptable derivatives include especially salts, including sulphates.

The active particles may be particles of ipatropium bromide.

The active particles may include a steroid, which may be beclomethasone dipropionate or may be Fluticasone. The active principle may include a cromone which may be sodium cromoglycate or nedocromil. The active principle may include a leukotriene receptor antagonist.

The active particles may include a carbohydrate, for example heparin.

The active particles may advantageously comprise a pharmacologically active agent for systemic use and advantageously they are capable of being absorbed into the circulatory system via the lungs. For example, the active particles may comprise peptides or polypeptides such as Dnase, leukotrienes or insulin. The pharmaceutical compositions of the invention may in particular have application in the administration of insulin to diabetic patients, preferably avoiding the normally invasive administration techniques used for that agent. The composite excipient particles could also be used for the administration of other agents for example for pain relief (e.g. analgesics such as Fentanyl or dihydroergotamine E which are used for the treatment of migraine), anti cancer activity, anti-virals, antibiotics or the delivery of vaccines to the respiratory tract.

Whilst the cholesterol may be added directly to the active particles, it is also possible to combine the cholesterol with particles of excipient material as a first step to form an excipient composition and then to mix that excipient composition with the active particles and with any other desired ingredients such as taste modifiers.

Accordingly, the invention also provides an excipient composition comprising particles of excipient material and cholesterol. The particles of excipient material may be fine excipient particles. The particles of excipient material may be carrier particles. The particles of excipient material may be a mixture of fine excipient particles and carrier particles.

The excipient composition preferably comprises not more than 60%, advantageously not more than 50%, more preferably not more than 30% and may, in some cases, comprise not more than 20% cholesterol by weight based on the combined weight of the cholesterol and the particles of excipient material. In general, the amount of cholesterol is at least 0.01% by weight based on the combined weight of the cholesterol and particles of excipient material.

The word “excipient” as used herein refers to any solid, substantially pharmaceutically inactive material which is acceptable for inclusion in pharmaceutical formulations material. The excipient material may, in particular, be composed of one or more materials selected from the materials stated above to be suitable for use as carrier particles.

The invention also provides a method of making a composition comprising active particles for use in an inhaler, the method comprising the step of mixing active particles with cholesterol. The active particles and the cholesterol may be mixed in any suitable way, for example, they may be blended together in a rotating blender or a Turbula mixer. Preferably, however, the active particles and the cholesterol are milled together, that is, the method includes a milling step which is described in more detail below.

The invention also provides a method of making a powder comprising active particles for use in a dry powder inhaler, the method comprising the step of mixing active particles with an excipient composition.

The invention further provides a method of making an excipient composition for use in a powder for inhalation comprising the step of mixing particles of an excipient material with cholesterol.

Where the particles of excipient material are carrier particles and it is not desired to reduce their size the mixing will typically be gentle, such as blending in a Turbula mixer. However, where it is intended that the particles of excipient material be fine excipient particles, the particles of excipient material (which may be fine or coarse) may be mixed with the cholesterol and the mixture may then be milled in a milling step.

Thus, preferably the active particles and/or particles of excipient material are milled in the presence of the cholesterol.

The word “milling” as used herein refers to any mechanical process which applies sufficient force to the active particles and/or the particles of excipient material that it is capable of breaking coarse particles (for example, particles of mass medium aerodynamic diameter greater than 100 μm) down to smaller particles of mass median aerodynamic diameter not more than 50 μm. For example, the milling step may be one which, if the active particles or the particles of excipient material were replaced with the same weight of lactose having a MMAD of between 150 and 200 μm, would be capable of reducing the MMAD of that lactose to below 50 μm. It has been found that processes which do not apply that degree of force, for example, blending, are not as effective at applying the additive material to the surfaces of the active or excipient particles. It is believed that is because that degree of force is required to separate the individual active particles and/or particles of excipient material such that effective mixing and effective application of the cholesterol to the surfaces of those particles is achieved. It should be understood, however, that in the case where the active particles and/or the particles of excipient material are already quite small, for example, having a mass median aerodynamic diameter below 60μ prior to the milling step, the size of those particles may not be significantly reduced. The important thing is that the milling process applies a sufficiently high degree of force or energy to the particles.

A wide range of milling devices and conditions are suitable for use in the milling step. The selection of appropriate milling conditions, for example, intensity of milling and duration, to provide the required degree of force will be within the ability of the skilled person who will understand how to arrange those milling conditions such that the milling is capable of breaking down coarse particles, as mentioned above. Ball milling is a preferred method. Alternatively, the milling step may utilize a high pressure homogenizer as described above. The milling step may, alternatively, involve an agitator bead mill, for example, the DYNO-mill (Willy A. Bachofen A G, Switzerland) or a Mechano-Fusion system (Hosokawa Micron Ltd) or a Hybridizer (Nara). Other possible milling devices include air jet mills, pin mills, hammer mills, knife mills and ultracentrifugal mills. Preferably, the milling process is a sealed process, preventing the escape of the cholesterol as fine particles.

Where active material or the excipient material is in the form of coarse particles prior to the milling step their size will be substantially reduced during the milling step.

Where excipient material is being milled in the presence of cholesterol the mass median aerodynamic diameter of the particles of excipient material is preferably not more than 50 μm, advantageously not more than 20 μm, more preferably not more than 15 μm and especially preferably not more than 10 μm after the milling step. Furthermore, 90% by weight of the excipient particles may have an aerodynamic diameter of less than 50 μm, advantageously less than 20 μm, more preferably less than 15 μm and especially preferably less than 10 μm. The mass median aerodynamic diameter of the milled particles will, in general not be less than 0.1 μm.

Where active particles are being milled in the presence of cholesterol the MMAD of the milled active particles is preferably not more than 10 μm, and advantageously it is not more than 5 μm, more preferably not more than 3 μm and in some cases may be not more than 1 μm. Accordingly, advantageously at least 90 by weight of the milled active particles have an aerodynamic diameter of not more than 10 μm, advantageously not more than 5 μm, preferably not more than 3 μm and more preferably not more than 1 μm. Advantageously, after the milling step, the active particles will be of a suitable size for inhalation to the desired part of the lung, for example, having an MMAD in the range of 5 to 0.5 μm for absorption in the respiratory bronchioles, 10 to 2 μm for delivery to the higher respiratory system and 2 to 0.05 μm for delivery to the alveoli. Accordingly, advantageously at least 90% by weight of the milled active particles have an aerodynamic diameter in the range of 5 to 0.5 μm, or of 10 to 2 μm, or of 2 to 0.05 μm. The MMAD of the active particles will not usually be lower than 0.01 μm.

The milling step may be carried out in a closed vessel, for example in a ball mill. The use of a closed vessel prevents loss of ultrafine particles or vapour of the cholesterol which has been found to occur in jet milling or other open processes. The milling may be dry, that is to say, there is no liquid present and the mixture to be milled is in the form of a dry particulate. Preferably, the milling is wet, that is, the milling step is carried out in the presence of a liquid. The liquid medium may be aqueous or non-aqueous, high or low volatility and of any solid content as long as it does not dissolve the active particles and/or particles of excipient material to any significant degree and its viscosity is not so high that it prevents motion of the balls. The liquid may also be one in which the cholesterol dissolves. Optionally, however, the cholesterol is not dissolved and is present in the form of particles. Where the cholesterol is soluble in the liquid medium it will be present as a solution during the milling step and will adsorb to the active or excipient particle surfaces. Advantageously, the liquid is then evaporated to leave the cholesterol on the surfaces of the active and/or excipient particles as solid cholesterol.

The presence of a liquid medium helps to prevent compacting of the mixture on the walls of the vessel and may also allow the more even spreading of the cholesterol on the surface of the active particles and/or the particles of excipient material as compared to dry milling. Preferably, the method also comprises the step of removing the liquid after the milling step. That may be accomplished by sieving followed by spray drying, or by evaporation of the liquid (followed by milling, if necessary, to break up large aggregates or cakes of material or by freeze drying). Preferably, the liquid is removed by spray drying.

As mentioned above, the milled active and/or excipient particles produced after the milling step may be of a suitable size for use in a pharmaceutical composition, for example, a powder or suspension for inhalation. However, it may also be desirable for the milled particles to be smaller than that and to have after the milling step an agglomeration step in which the milled particles are agglomerated to form agglomerated particles. In that way agglomerates of a size tailored to the requirement may be produced. Preferably, the agglomeration step is a spray drying step. The spray drying conditions may be selected to produce droplets having a desired size in the range of 1000 μm to 0.5 μm. The size of the agglomerates produced will depend largely on the concentration of the milled particles in the spray feed and the droplet size. Other materials, for example, binders may be included in the spray feed. Where the milling step is wet milling, the suspension or slurry may be spray dried directly after the milling step. Agglomeration may also be conducted in a fluid bed dryer or granulator.

Where cholesterol is lost in the process, for example, as particles carried away in the filtrate when a liquid milling medium is filtered off, it may be necessary to add more cholesterol at the start of the milling step than is desired in the final particles.

In a further aspect, the invention provides a dry powder inhaler comprising a composition as described above.

In a yet further aspect, the invention provides a pMDI comprising a composition as described above.

Embodiments of the invention will now be described for the purposes of illustration only, with reference to the following drawing, in which:

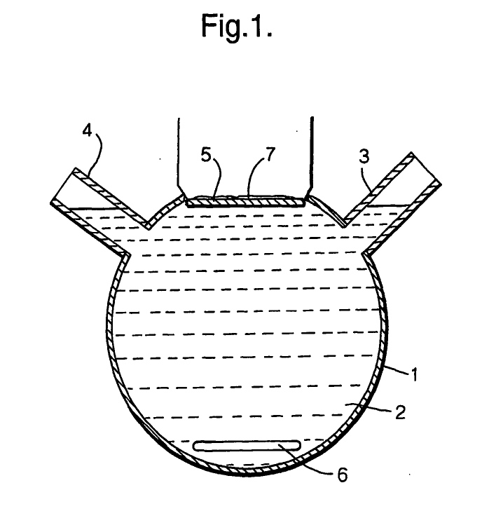

FIG. 1 shows an apparatus for use in the test described below.

Evaluation of the fine particle fraction of multi-stage liquid impinger (MSLI) was carried out in accordance with method given in European Pharmacopoeia, Supplement 2000, Section 2.9.18.

Evaluation of the fine particle fraction using a twin stage liquid impinger (TSI) was carried out in accordance with the method of WO 96/23485 pages 29 to 33. The method is also described in the European Pharmacopoeia referred to above and in J. Pharm. Pharmacol, 1987, 39, 966-972).

The test used as a model test for the length of time taken for a particular formulation to dissolve on the lung membrane is as follows.

The apparatus used is shown in FIG. 1 and comprises a 195 cm³ reservoir (1) filled with deionised water (2) and having an inlet port (3) and an outlet port (4). A sintered glass disc (5) of approximately 50 mm diameter and 3 mm depth occupies an opening at the top of the reservoir (1) and sits horizontally in contact with the water (2). The water in the reservoir is stirred by a magnetic stirrer (6).

A known mass of approximately 1 mg of the formulation (7) to be tested is placed on the sinter and a timer is started. At various times, 1 cm³ samples of the water are removed from the reservoir and are immediately replaced with 1 cm³ deionised water to maintain the volume in the reservoir. The concentration of the active substance in the 1 cm³ samples is determined by a suitable method. The particular method will, of course, depend on the nature of the active substance but such methods will be known to the skilled person.

A graph of concentration of the active substance in the reservoir of water versus time is then plotted.

The degree to which the cholesterol delays the dissolution of the active substance is a measure of the likely delay of release of the active substance in the lung. In particular, where it is desired to measure the delayed release performance of a particular composition, that can be done by carrying out the standard dissolution test given above on a sample of the composition to be tested and upon a (control) sample of the active substance. For a true comparison the particle size distribution of the particles of the active substance must be the same or similar in the sample of composition to be tested as in the (control) sample of active substance. The rate of dissolution of the active substance in the microparticles to be tested as a percentage of the rate of dissolution of the active substance alone can then be calculated by the following formula:

$$\% rate of dissolution = \frac{TA}{TF} \times 100$$

Where TA=time taken for the concentration of the active substance to reach a maximum for the sample of active substance alone.

Where TF=time taken for the concentration of the active substance to reach a maximum for the sample of the composition to be tested.

Thus, for example, if the concentration of the active substance in the dissolution test on the composition comprising cholesterol reached a maximum at 40 minutes and the concentration of the active substance alone reached a maximum at 10 minutes, the % rate of dissolution for the formulation would be 10/40×100=25%, corresponding to a decrease in the rate of dissolution of 75%.

### EXAMPLE 1

1 g of cholesterol, 10 g of lactose having a MMAD of approximately 8 μm (Microfine manufactured by Borculo) and 10 cm³ cyclohexane were combined in a ball mill and milled for 90 minutes in the presence of 50 g of 5 mm diameter milling balls. The resulting paste was left open to the atmosphere and the cyclohexane was allowed to evaporate overnight. The resulting powder was milled again briefly to give an excipient powder.

0.5 g of the excipient powder, 0.5 g salbutamol sulphate and 4 g of a large carrier lactose were tumbled together for 30 minutes to produce a powder suitable for inhalation.

The powder was fired from a Cyclohaler into a twin stage impinger at a flow rate of 60 litres/min (1 pm). The average fine particle fraction obtained was 55%.

### EXAMPLE 2

1 g of cholesterol, 1 g of lecithin, 10 g of lactose having a MMAD of approximately 8 μm (Microfine, manufactured by Borculo) and 10 cm³ dichloromethane were combined in a ball mill and milled for 90 minutes in the presence of 50 g of 5 mm diameter milling balls. The resulting paste was left open to the atmosphere overnight to evaporate the dichloromethane. The resulting material was milled briefly to give an excipient powder.

The excipient powder was blended with a large carrier lactose and micronised salbutamol sulphate. This formulation was fired from a Cyclohaler into a twin stage impinger at 60 1 pm. The average fine particle fraction obtained was 65%.

### EXAMPLE 3

2 g of cholesterol and 98 g of lactose were milled together in a Retsch S100 centrifugal mill for 30 minutes at 580 rpm in the presence of 300 g of stainless steel milling balls having diameters in the range of from 3 to 10 mm to give an excipient powder.

### EXAMPLE 4

0.5 g of cholesterol and 5 g of micronised salbutamol sulphate (particle size distribution 1 to 5 μm) were combined with 124 g of 3 mm stainless steel balls in a steel milling vessel and were milled at 500 rpm in a Retsch S100 centrifugal mill for 5 hours. The powder was dried and sieved to remove the steel balls to give a composition suitable for inhalation. Examination of the powder with a scanning electron microscope revealed that the particles had diameters in the range of 0.1 to 0.5 μm.

A high pressure homogeniser or bead mill may be used in place of the ball mill.

### EXAMPLE 5

Cholesterol and Microfine lactose in a ratio of 1:10 by weight were ball milled as a suspension in dichloromethane for 90 minutes. The suspension was then dried and the resulting powder recovered. This powder was blended in a Turbula mixer with a large carrier lactose and micronised budesonide. This formulation was fired from a Cyclohaler into a multistage liquid impinger at 90 lpm. The average fine particle fraction obtained was 46%.

### EXAMPLE 6

Cholesterol powder was jet milled. The resulting powder was then blended with Microfine lactose in a high shear blender. This powder was blended with a coarse carrier lactose and micronised budesonide. The example was repeated twice, once using a Hosokowa Mechanofusion system and once using a Passcal ball mill in place of the high shear blender.

### EXAMPLE 7

Cholesterol powder was jet milled. The resulting powder was then blended with micronised budesonide in a Hosokawa Mechanofusion system. This powder was then blended with a large carrier lactose.

### EXAMPLE 8

Cholesterol powder was processed as a suspension in water in a high pressure homogenizer. A fine cholesterol powder was recovered. That powder was then blended with Microfine lactose in a high shear blender. The resulting mixture was blended with a large carrier lactose and micronised budesonide. The example was repeated twice, once using a Hosokawa Mechanofusion system and once using a Passcal ball mill in place of the high shear blender.

### EXAMPLE 9

Cholesterol powder was processed as a suspension in water in a high pressure homogenizer. A fine powder was recovered. That powder was then blended with micronised budesonide in a Hosokawa Mechanofusion system. The resulting mixture was then blended with a large carrier lactose in a Turbula mixer.

It was observed in some cases that when ball milling active particles with cholesterol, a fine powder was not produced. Instead the powder was compacted on the walls of the mill by the action of the mill. That inhibited the milling action and prevented the effective mixing of the active and cholesterol. That problem occurred particularly where the cholesterol was present in small proportions (typically <2%), in cases where the milling balls were relatively small (typically <3 mm), in cases where the milling speed was too slow and where the starting particles were too fine. To prevent this occurring it is advantageous to mill in a liquid medium. The liquid medium reduces the tendency to compaction, assists the distribution of the cholesterol and improves any milling action.

It has been found to be preferable to use a large number of fine milling balls, rather than fewer heavy balls. The finer balls perform a more efficient co-milling action. Preferably the balls have a diameter of less than 5 mm, advantageously less than 2 mm. Liquid media are preferred which are non flammable, for example dichloromethane and fluorinated hydrocarbons, especially fluorinated hydrocarbons which are suitable for use as propellants in inhalers.

A particularly preferred method is milling using a high pressure homogeniser, as this reduces contamination as compared to ball milling, for example, where the collisions between the balls may produce contaminants.

In the wet milling process, the cholesterol appears to confer several advantages: it allows the milling process to be more efficient, with smaller particles produced and compaction reduced, the particles may be stabilised in suspension, and on drying, the cholesterol remains as a coating around the particles which may aid dispersion, and may modify the subsequent dissolution characteristics of the particle.

When the active material is a protein, the milling may be preceded by lyophilisation (freeze drying) of the protein either pure or in combination with an additive material and/or a polymeric stabliser. The freeze drying may make them more brittle and more easily milled. The milling may be conducted under cryogenic (cold) conditions to make the particles more brittle.

1. A composition for use in an inhaler comprising active particles and cholesterol particles.

2. A composition as claimed in claim 1, which also comprises a propellant and is suitable for use in a pressurised metered dose inhaler.

3. A composition as claimed in claim 1, which is a powder for use in a dry powder inhaler.

4. A composition as claimed in claim 3, which comprises not less than 40% by weight of the active particles based on the weight of the total composition.

5. A composition as claimed in claim 3 or claim 4, which comprises not more than 60% cholesterol by weight based on the weight of the total composition.

6. A composition as claimed in any preceding claim, in which at least some of the cholesterol is present on the surfaces of the active particles.

7. A composition as claimed in any preceding claim, which also comprises fine excipient particles.

8. A composition as claimed in any preceding claim, which also comprises carrier particles.

9. A composition as claimed in any preceding claim, which also comprises carrier particles and/or fine excipient particles and the cholesterol is present on the surfaces of the carrier particles and/or the fine excipient particles.

10. A composition as claimed in any preceding claim, in which the cholesterol promotes the dispersal of the active particles on actuation of the inhaler.

11. A composition as claimed in any preceeding claim being such that, upon inhalation of the composition, the active substance exerts its pharmacological effect over a period significantly greater than the period over which the active substance exerts its pharmacological effect when inhaled alone.

12. A composition as claimed in any preceding claim wherein the rate of dissolution of the active substance is no greater than 80% of the rate of dissolution of particles of the active substance alone.

13. An excipient composition for use in a powder for inhalation, the excipient composition comprising particles of excipient material and cholesterol.

14. An excipient composition as claimed in claim 13, in which the particles of excipient material are fine excipient particles.

15. An excipient composition as claimed in claim 13 in which the particles of excipient material are carrier particles.

16. An excipient composition as claimed in claim 13, in which the particles of excipient material are a mixture of fine excipient particles and carrier particles.

17. An excipient composition as claimed in any of claims 13 to 16, in which the cholesterol is present in an amount of not more than 60% by weight based on the combined weight of cholesterol and of excipient material.

18. An excipient composition as claimed in any of claims 13 to 17, in which the particles of excipient material are of lactose.

19. Particles of cholesterol having a diameter of 2 μm or less.

20. Particles of cholesterol as claimed in claim 19 having a diameter of 1.5 μm or less.

21. A method of making a composition comprising active particles for use in an inhaler, the method comprising the step of mixing active particles with cholesterol.

22. A method as claimed in claim 21, in which the active particles and the cholesterol are milled together.

23. A method of making a powder comprising active particles for use in a dry powder inhaler, the method comprising the step of mixing active particles with an excipient composition as claimed in any of claims 14 to 20.

24. A method of making an excipient composition for use in a powder for inhalation comprising the step of mixing particles of an excipient material with cholesterol.

25. A method as claimed in claim 22, in which the particles of excipient material and the cholesterol are milled together.

26. A dry powder inhaler comprising a composition as claimed in any of claims 3 to 13.

27. A pressurised metered dose inhaler comprising a composition as claimed in any of claims 1 or 2 or 4 to 6.

28. Use of cholesterol in a composition for inhalation comprising active particles, for promoting the dispersal of the active particles to form an aerosol on actuation of the inhaler.

29. Use of cholesterol in a composition for inhalation comprising active particles, to delay the dissolution of the active substance in the lung.

30. Use as claimed in claim 28 or claim 29, in which the composition is a dry powder for use in a dry powder inhaler.

31. A composition substantially as described herein in Examples 1 to 9.

32. An excipient composition substantially as described herein.
